(19) United States
(12) Patent Application Publication
(10) Pub. No.: US 2017/0013860 A1
(43) Pub. Date: Jan. 19, 2017

(54) PRODUCTS CONTAINING PARTIALLY HYDROLYZED SOY BETA-CONGLYCININ, AND RELATED METHODS

(71) Applicant: PURDUE RESEARCH FOUNDATION, West Lafayette, IN (US)

(72) Inventors: Pui Yeu Phoon, Camby, IN (US); Maria Fernanda San Martin-Gonzalez, West Lafayette, IN (US); Narsimhan Ganesan, West Lafayette, IN (US)

(73) Assignee: PURDUE RESEARCH FOUNDATION, West Lafayette, IN (US)

(21) Appl. No.: 15/052,913

(22) Filed: Feb. 25, 2016

Related U.S. Application Data
(63) Continuation of application No. 14/246,781, filed on Apr. 7, 2014, now abandoned.
Application No. 14/246,781 is a continuation of application No. PCT/US2012/059034, filed on Oct. 5, 2012.
(60) Provisional application No. 61/544,866, filed on Oct. 7, 2011.

(51) Int. Cl. A23J 3/16; A23J 1/14; A23L 33/12; A61K 9/50; A23L 33/185; A61K 47/42; A61K 9/107; A23D 7/005; A23J 3/30

(52) U.S. Cl. CPC A23J 3/16; A23D 7/0053; A23J 1/14; A23L 33/12; A23J 3/30; A23L 33/185; A61K 47/42; A61K 9/107; A61K 9/5052; A23V 2002/00

(57) ABSTRACT

Described are emulsion compositions comprising oil, water and partially hydrolyzed soy beta-conglycinin, as well as materials and methods for their preparation and use. The soy beta-conglycinin can be enzyme-hydrolyzed material, such as trypsinized material. The degree of hydrolysis of the soy beta-conglycinin can be light, for example up to 2.5%. The hydrolyzed soy beta-conglycinin can be effective to form fibril sheets adsorbed to oil droplets at the interface between the droplets and a continuous aqueous phase in an emulsion composition. The soy beta-conglycinin can be hydrolyzed to such an extent that it provides improved oxidative stability to the oil in the emulsion composition while also providing physical stability equal to and/or greater than that obtained using a corresponding nonhydrolyzed soy beta-conglycinin composition.

### RELATED APPLICATION

The present application is a continuation of U.S. application Ser. No. 14/246,781 filed Apr. 7, 2014, which claims the benefit of priority to International App. No PCT/US/US2012/059034 filed Oct. 5, 2012 which claims the benefit of U.S. Prov. App. No. 61/544,866, filed Oct. 7, 2011, the disclosures of each of which is hereby incorporated by reference in its entirety.

### BACKGROUND

The present invention relates generally to products useful as or in foods, food additives, or medical products, and in certain of its aspects to such products containing water, oil, and a partially hydrolyzed soy bean beta-conglycinin material.

As further background, soy bean protein isolates or concentrates and certain hydrolysates thereof have been investigated as ingredients of food, medical and other products. The glycinin (11S) and β-conglycinin (7S) globulins constitute 36-53% and 30-46%, respectively, of the total water-extractable proteins in soy, making them the two most abundant storage proteins (Saio et al., 1969). At physiological pH, the molecular masses of 11S and 7S are very large at 350 kDa (Badley et al., 1975) and 180-210 kDa (Koshiyama, 1968), respectively. Due to the large size and compact globular structure, the rate of diffusion onto an adsorption surface and in turn the rate of increase in surface pressure is expected to be slow, without significant interfacial denaturation (Santiago et al., 2008). Consequentially, emulsifying activity is expected to be poor.

The reduction of size may improve adsorption and interfacial properties. Ryan et al. (2008) reported a higher emulsion thermal stability observed in soy protein isolate than in commercial soy protein hydrolysates of unknown degree of hydrolysis (DH). However, other researchers (Ruíz-Henestrosa et al., 2007; Martínez et al., 2009; Ruíz-Henestrosa et al., 2007) showed that limited hydrolysis at less than 6% DH improved the surface activity of soy proteins, when non-specific enzymes such as fungal protease and the bacterial protease Alcalase were used.

There remain needs for improved and/or alternative soy protein derived compositions that display beneficial properties in the preparation, characteristics and/or maintenance (e.g. oxidative and/or storage stability) of products containing them, including oil/water emulsion products. The present invention is addressed to these needs and in certain preferred aspects involves the use of modified soy bean beta-conglycinin as the sole component providing both physical and oxidative stability in oil in water emulsion compositions.

### SUMMARY

In certain aspects, the present invention relates to compositions comprising oil in water emulsions, and methods for their preparation and/or use, wherein the compositions include partially hydrolyzed soy bean beta-conglycinin. One, some, or all of the following additional features can be included in the compositions and/or methods:

the degree of hydrolysis of the partially hydrolyzed soy bean beta-conglycinin can be up to about 5%, preferably up to about 2.5%;

the soy bean beta-conglycinin can be enzymatically hydrolyzed, preferably by trypsin;

the oil in the emulsion can include omega-3 fatty acids, for example included in or derived from fish oil;

the partially hydrolyzed soy bean beta-conglycinin can constitutes less than about 1% (w/v), or less than about 0.5% (w/v) of the composition;

the soy bean beta-conglycinin can be partially hydrolyzed to such an extent that the oil in water emulsion has an oxidative stability greater than, and a physical stability at least equal to, a corresponding oil in water emulsion prepared with a corresponding nonhydrolyzed soy bean beta-conglycinin;

the degree of hydrolysis of the partially hydrolyzed soy bean beta-conglycinin can be up to about 1.5%;

the degree of hydrolysis of the partially hydrolyzed soy bean beta-conglycinin can be about 0.5% to about 1%;

the composition can be a food product composition containing at least one member selected from the group consisting of a flavorant, a colorant, a source of protein other than the partially hydrolyzed soy beta-conglycinin, and a source of carbohydrate; or at least two members of this group; or all members of this group.

the partially hydrolyzed soy bean beta-conglycinin can form fibril sheets adsorbed to oil droplets in the emulsion;

the partially hydrolyzed soy bean beta-conglycinin can increase the stability of the oil to oxidation as compared to a corresponding nonhydrolyzed soy bean beta-conglycinin;

the initial oxidation (over the first 24 hours) of oil encapsulated by partially hydrolyzed soy bean beta-conglycinin can be slowed or reduced by at least about 5 fold, or at least about 8-fold, as compared to a corresponding emulsion with corresponding nonhydrolyzed soy bean beta-conglycinin.

In another embodiment, provided is a method for preparing an emulsion composition, comprising emulsifying a mixture including water, oil, and partially hydrolyzed soy bean beta-conglycinin. The characteristics (e.g. degree of hydrolysis) of the partially hydrolyzed soy bean beta-conglycinin, as well as the ingredients and relative amounts of the ingredients used to prepare the emulsion, can be selected from among any of those identified above or elsewhere herein.

Additional embodiments of the invention relate to the use of compositions of the invention as, or in the preparation of, a food product, a food additive for fortification of another food, or a medical product (e.g. administrable orally or parenterally).

Still further embodiments as well as features and advantages of aspects of the invention will be apparent from the descriptions herein.

### BRIEF DESCRIPTION OF THE DRAWINGS

FIG. 1 shows plots showing the degree of hydrolysis (DH) in 0.5% (w/v) soy 7S hydrolysates (7SH) in 0.02 M disodium phosphate buffer, as a function of (1) Enzyme/substrate (E/S) ratio (w/w) during trypsinization; and (2) Incubation time during acid hydrolysis, as further described in the Specific Experimental below. Error bars indicate the standard deviation from the mean of three measurements.

FIG. 2 shows plots of relative intrinsic rate of oxygen depletion in encapsulated fish oil per unit interfacial area versus time, for an emulsion stabilized by a soy 7S hydrolysate from acid hydrolysis at pH 3, as compared to an emulsion stabilized by the native 7S control at pH 7, as further described in the Specific Experimental below. The protein concentration used was 0.5% (w/v), in 0.02 M disodium phosphate buffer. Error bars indicate the standard deviation from the mean of four readings from duplicates.

FIG. 3 shows the characterization of emulsions stabilized by 0.5% (w/v) trypsinized 7S in 0.02 M disodium phosphate buffer at pH 7, as further described in the Specific Experimental below. (Panel 1) Plots of change in d₃₂ of emulsions with time. Insert: Change in count rate of the emulsion drops measured during dynamic light scattering experiment. (Panel 2) Plots of relative intrinsic rate of oxygen depletion in encapsulated fish oil per unit interfacial area versus time, for emulsions stabilized by trypsinized 7S compared to the native 7S control and the heated 7S control. (Panel 3) Plots of relative intrinsic rate of oxygen depletion in encapsulated fish oil per unit interfacial area versus time, for emulsions stabilized by trypsinized 7S of different DH compared to the milk proteins sodium caseinate and P-lactoglobulin. Error bars indicate the standard deviation from the mean of four readings from duplicates.

FIG. 4 shows the characterization of emulsions stabilized by 0.2% (w/v) trypsinized (Tryp) 7S as compared to 0.5% (w/v) trypsinized 7S, in 0.02 M disodium phosphate buffer at pH 7, as further described in the Specific Experimental below. The degree of hydrolysis (DH) was 0.7%. (Panel 1) Plots of change in d₃₂ of emulsions with time. (Panel 2) Plots of relative intrinsic rate of oxygen depletion in encapsulated fish oil per unit interfacial area versus time. Error bars indicate the standard deviation from the mean of four readings from duplicates.

FIG. 5 shows the characterization of emulsions stabilized by 0.5% (w/v) trypsinized (Tryp) 7S in different ionic strengths of disodium phosphate buffer at pH 7, 5 as further described in the Specific Experimental below. The degree of hydrolysis (DH) was 0.7%. (Panel 1) Plots of change in d₃₂ of emulsions with time. The zeta potential of each solution was indicated. (Panel 2) Plots of relative intrinsic rate of oxygen depletion in encapsulated fish oil per unit interfacial area versus time. Insert: Plots showing the effect of ionic strength on PV. Error bars indicate the standard deviation 10 from the mean of four readings from duplicates.

FIG. 6 shows the characterization of emulsions stabilized by 0.5% (w/v) trypsinized (Tryp) 7S in 0.02 M disodium phosphate buffer, at different pH values, as further described in the Specific Experimental below. The degree of hydrolysis (DH) was 0.7%. (Panel 1) Plots of change in d₃₂ of emulsions with time. (Panel 2) Plots of relative intrinsic rate of oxygen depletion in encapsulated fish oil per unit interfacial area versus time. *The emulsion stabilized by DATEM in addition to 7SH was also characterized. Error bars indicate the standard deviation from the mean of four readings from duplicates.

FIG. 7 provides Raman spectra of two models at 2% (w/v) protein 20 concentration within the 1500-1750 cm⁻¹ frequency range, as further described in the Specific Experimental below. Each spectrum was an average of three scans at different points.

FIG. 8 provides Raman spectra of 2% (w/v) 7SH from trypsinization and the native 7S control at the hydrophobicized silver surface, within the 1500-1750 cm⁻¹ frequency range, as further described in the Specific Experimental below. The spectra were collected immediately after depositing the protein solutions on the silver. *Exception: Raman spectrum of less-than-2% (w/v) 7SH from trypsinization at an anionic silver surface, with the intensity scaled to that at 2% for comparison purpose. Each spectrum was an average of three scans at different points.

FIG. 9 provides Raman spectra of 2% (w/v) 7SH from trypsinization and the native 7S control at the hydrophobicized silver surface, within the 1500-1750 cm-1 frequency range, as further described in the Specific Experimental below. The spectra were collected 1 day after depositing the protein solutions on the silver. *Exception: Raman spectrum of less-than-2% (w/v) 7SH from trypsinization at an anionic silver surface, with the intensity scaled to that at 2% for comparison purpose. Each spectrum was an average of three scans at different points.

### DETAILED DESCRIPTION

For the purpose of promoting an understanding of the principles of the invention, reference will now be made to certain embodiments and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

As disclosed above, certain aspects of the present invention provide compositions, including oil in water emulsion compositions, which comprise a partially hydrolyzed soy bean beta-conglycinin material. In conjunction with the description of embodiments in the present Detailed Description, it will be understood that any of the features identified in the Summary above, or any combination of some or all of such features, may be present in the described embodiments. All such combinations are contemplated as embodiments disclosed herein.

Embodiments of the invention utilize partially hydrolyzed soy bean beta-conglycinin material. For these purposes, the soy bean beta-conglycinin material can be obtained by any suitable method, including for example from native or genetically modified plants. Partial hydrolysis of the beta-conglycinin can be accomplished in any suitable fashion, with the use of enzymes to accomplish at least a part of, and in some embodiments all of, the hydrolysis, being preferred. Suitable enzymes for hydrolysis, such as proteases, are known. Trypsin is a preferred enzyme for these purposes.

The degree of hydrolysis of the beta-conglycinin is preferably low, for example up to about 2.5%. In this regard, “degree of hydrolysis” (DH) as used herein means the value calculated as:

$$DH ( \% ) = \frac{{h}_{eqv}}{{h}_{total}} \times 100 \qquad ( 4 )$$

where h_{eqv} was the amount of peptide bonds cleaved in equivalent (meqv/g of protein), and hₜₒₜₐₗ, estimated at 9.1, was the sum of number of amino acid residues in the heterotrimer molecules per gram of soy 7S; further description is also found in Section 2.11 of the Experimental below. In additional embodiments, the degree of hydrolysis can be in the range of about 0.1% to about 2.5%, or about 0.1% to about 1.5%, or about 0.5% to about 1.5%. Alternatively or additionally, the degree of hydrolysis can be controlled so that the partially hydrolyzed beta-conglycinin has a beneficial property or properties disclosed herein. As examples, these properties can involve the ability to form beta-sheet fibrils at the oil/water interface of oil in water emulsions, preferably encapsulating oil droplets thereof, and/or the ability to physically stabilize an oil in water emulsion at a level least as well as, and preferably greater than, the corresponding nonhydrolyzed beta-conglycinin material, and/or the ability to stabilize the oil in an oil in water emulsion against oxidation to an extent greater than the corresponding nonhydrolyzed beta-conglycinin. Methodologies for assessment of these parameters are found in the specific Experimental below.

Compositions of the invention can be constituted at any suitable percentage by weight of the partially hydrolyzed beta-conglycinin. For example, compositions that are purified or enriched in the partially hydrolyzed beta-conglycinin can be constituted at least about 95% by weight, or at least about 99% by weight of the partially hydrolyzed beta-conglycinin, and can be provided for instance as dry powders. Compositions that consist, or consist essentially of, the partially hydrolyzed beta-conglycinin (e.g. as dry powders) are also contemplated. Oil in water emulsion compositions that include the partially hydrolyzed beta-conglycinin can be constituted to a relatively low level by the partially hydrolyzed beta-conglycinin, for example less than about 5% weight/volume (w/v), less than about 3% weight/volume, less than about 1% weight/volume, or less than about 0.5% weight/volume. Other levels of the partially hydrolyzed beta-conglycinin could also be used depending on other factors, including for example the amount of oil in the emulsion relative to water. The ratio of water to oil in such emulsions can be any suitable ratio, including for example volume:volume (v/v) ratios in the range of about 70:30 to about 30:70. Emulsions having a greater volume of water than oil are provided in certain embodiments. Illustratively, the emulsion can have a v/v ratio of oil to water of less than about 30:70, less than about 10:90, or less than about 5:95 in some inventive variants. Oils that contain essential omega-3 unsaturated fatty acids are preferred. These may include, for example, one or more oils derived from fish, plants or parts thereof (e.g. walnuts or flax seed), or algae (e.g. golden marine algae). In certain embodiments, oil in water emulsion compositions are provided that are free from animal-derived substances, or at least free from animal-derived fats, oils and/or proteins, which can for example be used as foods or nutritional fortifiers or supplements.

Additionally or alternatively, the partially hydrolyzed beta-conglycinin can constitute any suitable percentage by weight of the total soy bean protein in the composition. Compositions in which the partially hydrolyzed beta-conglycinin constitutes at least 50%, at least 70%, at least 90%, at least 95%, or at least 99% by weight of the total soy bean protein in the composition are provided in embodiments herein. Also, compositions in which the total soy bean protein in the composition consists, or consist essentially, of the partially hydrolyzed beta-conglycinin, are contemplated herein.

The compositions of the invention can, for example, be provided as foods, food additives such as fortifiers, or medical compositions (e.g. for oral or parenteral administration for nutrition or other purposes). Food compositions of the invention may contain other ingredients conventions thereto, including for example flavoring agents, coloring agents, one or more other sources of protein, one or more sources of carbohydrates, preservatives, and the like. Food compositions of the invention may, for example, be drinks (e.g. soft drinks, dairy milk or soy milk drinks), salad dressings (spoonable or poorable), whipped toppings, spreads, frozen desserts, or other similar products that comprise emulsions of oil and water. Food or medical compositions of the invention may be sterilized, and medical compositions may contain other pharmaceutically acceptable carriers or ingredients. Any oil/water emulsion composition of the invention described herein may optionally also utilize the soy bean beta-conglycinin as the sole agent present in the composition providing physical stability of the emulsion and/or oxidative stability to the oil, and thus such compositions can be free from other emulsifying agents and/or antioxidant agents. It will be understood, however, that the oxidative or physical stability provided by the hydrolyzed soy bean beta-conglycinin may be supplemented by other agents. In addition or alternatively, any oil/water emulsion composition of the invention described herein may optionally also contain an anionic additive to provide an anionic surface to the oil droplets to facilitate beta-fibril formation by the partially hydrolyzed beta-conglycinin at the oil/water interface.

### SPECIFIC EXPERIMENTAL

In order to promote a further understanding of the present invention and its features and advantages, the following experimental description is provided. It will be understood, however, that this experimental description is illustrative, and not limiting, of the invention.

### 1. ABSTRACT OF THE EXPERIMENTAL

In this study, the effect of limited hydrolysis of soy β-conglycinin (7S) on the oxidative stability of 7S hydrolysate (7SH)-stabilized emulsions was investigated. Two different methods of hydrolysis were carried out, namely trypsinization and acid hydrolysis. Menhaden oil-in-water emulsions (2%, v/v) were created via homogenization at 20 kpsi, under different conditions of pH, ionic strength, degrees of hydrolysis (DH), and protein concentration in the continuous phase. Oxidation of the emulsions was accelerated at 55° C. in the dark over 7 days, and monitored by the ferric thiocyananate peroxide value assay. The 7S and 7SH conferred oxidative stability in the following order (from worst to best): 7SH from acid hydrolysis <trypsin 7SH of 0.7% DH at pH 3<native 7S at pH 7<trypsin 7SH of 0.7% DH at pH 7<trypsin 7SH of 2.5% DH at pH 7<trypsin 7SH of 0.7% DH at pH 9<trypsin 7SH of 0.7% DH at pH 12.5. Among the main experimental findings was that acid hydrolysis yielded abundant β-sheet fibrils in the continuous aqueous phase, which adsorbed poorly onto the oil-water interface and thus caused poorer emulsion oxidative stability than the native 7S control. For trypsin hydrolysates, formation of fibrils was induced at the oil-water interface, and enhanced emulsion oxidative stability was observed at pH 7, and more so at pH 9 and 12.5. Results from Raman spectroscopy suggested an assembly of extended β-sheets close to the oil surface at alkaline pH, which probably reinforced the protection of oil in addition to protein unfolding that promoted protein-oil hydrophobic interaction. In contrast, the lack of improvement in oxidative stability with trypsin 7SH at pH 3 was attributed to limited interfacial unfolding of the acidic molten globule and a different alignment of fibrils.

### 2. MATERIALS AND METHODS

### 2.1 Chemicals

Defatted soy flour was purchased from Hodgson Mill. The following were purchased from Sigma-Aldrich Company: 1-anilino-8-naphthalene (ANS), 2-propanol, 30% hydrogen peroxide, 5% 2,4,6-trinitrobenzenesulfonic acid solution (TNBS), ammonium thiocyanate, barium chloride dehydrate, disodium phosphate, ferric chloride hexahydrate, ferrous sulfate heptahydrate, isooctane, Menhaden fish oil, potassium hydroxide, silver nitrate, sodium chloride, sodium lauryl sulphate (SDS), thioflavin T-dye, and trypsin from porcine pancreas with a declared activity of 1000-2000 BAEE units/mg. The following were purchased from Mallinckrodt Chemicals Inc.: butanol, hydrochloric acid, and methanol. Hexamethyldisilazane (HMDS) and sodium mercaptoethanesulfonate (MES) were purchased from TCI America. Iron powder was purchased from Acrõs Organics. Diacetyl tartaric acid ester of mono-diglycerides (DATEM) (Panodan® FDP K) was supplied by Danisco USA Inc. Other chemicals used included concentrated ammonium hydroxide and sodium azide from J.T. Baker, sodium bisulfite from Fisher Scientific, and glucose from A.E. Staley Manufacturing Company. Triple distilled water was used to prepare all aqueous solutions.

### 2.2 Purification of Soy 7S

The procedure for purification of soy 7S was adapted from Howard et al. (1983). The defatted soy flour used had a declared protein content of 14 g per 30 g flour. The soy flour (5%, w/v) was dissolved in water at pH 8 overnight, after which the mixture was centrifuged at 500×g for 15 min at 20° C. To the total volume of supernatant, 0.03 M of sodium chloride and 0.77 mM sodium bisulfite was added and the pH adjusted to 6 using ≦5 N hydrochloric acid and ≦5 N sodium hydroxide. This pH 6 mixture was centrifuged at 500×g for 15 min at 20° C., and the supernatant was adjusted to pH 5.5. The pH 5 supernatant was subjected to another round of centrifugation, and the supernatant was adjusted to pH 4.5. The pH 4.5 supernatant was subjected to a final round of centrifugation, and the precipitate was redissolved in water at pH 7. This was followed by overnight dialysis of the redissolved protein in triple distilled water at 4° C., using a Spectra/Pore 6 standard regenerated cellulose dialysis membrane of 50 kDa molecular weight cut-off. The dialyzed protein was freeze-dried and stored at −20° C. until use.

### 2.3 Hydrolysis of Soy 7S

Soy 7S (0.5%, w/v) in 0.02 M disodium phosphate solution and 0.02% (w/v) sodium azide was prepared for hydrolysis. As a control without hydrolysis, the protein solution was adjusted to pH 7 using ≦5 N hydrochloric acid and ≦5 N sodium hydroxide. Soy 7S protein solution (1%, w/v) in the same buffer constitution was also prepared for trypsinization, to be used specially in Section 2.4.2. In certain instances, the ionic strength of the buffer was 0.05 or 0.10 M disodium phosphate, and the protein concentration was 0.2% (w/v).

2.3.1 Acid Hydrolysis

The 7S protein solution was adjusted to pH 2 using ≦5 N hydrochloric acid, and heated at 82° C. in a shaking water bath at 200 rpm from 8 to 26 h.

2.3.2 Trypsinization

The 7S protein solution was adjusted to pH 8 using ≦5 N hydrochloric acid and ≦5 N sodium hydroxide, and trypsin was added in the enzyme/substrate ratio (w/w) of 0.04, 0.08, 0.12, or 0.15. The solution was incubated at 37° C. in a shaking water bath at 200 rpm for 6.5 h. Inactivation of the enzyme was accomplished by immersing the solution in a separate 80° C. water bath for 10 min followed by immediate cooling in water to room temperature. A second control was protein solution at pH 7 without trypsin added but also treated in the 80° C. water bath.

### 2.4 Emulsion Preparation

2.4.1 Emulsions with a Non-Polar Oil Surface

The 7S control (both treated and not treated in the 80° C. water bath as mentioned in Section 2.3) or hydrolysate was used to create 2% (v/v) fish oil-in-water emulsions via high pressure homogenization (HPH). The hydrolysates from acid hydrolysis were used at pH 3 and 7, while the trypsinized hydrolysates were used at pH 3, 7, 9, and 12.5. The emulsion was created first by coarse homogenization using a VirtiShear homogenizer at 30000 rpm for 10 s, immediately followed by HPH using a Nano DeBEE 45 high pressure homogeniser (BEE International) under 3 passes at 20 kpsi (137.9 MPa). For temperature control during HPH, a counter-current tubular heat exchanger connected to a 2° C. water bath was positioned downstream of the emulsifying cell. Each emulsion sample was prepared in duplicate.

2.4.2 Emulsions with an Anionic Oil Surface

To a 0.02 M disodium phosphate buffer with 0.02% (w/v) sodium azide at pH 3 was added Menhaden oil at 4% (v/v) and DATEM at 0.1% (w/v). The mixture was heated for 2 min in a 65° C. water bath, above the dropping temperature point of DATEM, then immediately subjected to coarse homogenization using the VirtiShear homogenizer at 30000 rpm for 10 s. This was followed promptly by HPH at 20 kpsi under 3 passes at 50° C. (above the dropping point of DATEM). The emulsion was cooled and the 1% (w/v) trypsin hydrolysate at pH 3 was added in a 1:1 (v/v) ratio. This mixture was again subjected to the VirtiShear homogenizer and eventually to HPH at 2° C., at 20 kpsi under 3 passes. The final emulsion was comprised of 0.5% (w/v) protein and 2% (v/v) oil. Each emulsion sample was prepared in duplicate.

### 2.5 Ferric Thiocyanate Peroxide Value Assay

1 ml emulsion volumes were pipetted into 1.775-ml screw-capped microcentrifuge tubes and incubated in a shaker in the dark at 55° C. Triplicate tubes were removed daily over a period of seven days to determine the oxidative stability, based on the spectrophotometric measurement of the ability of peroxides to oxidize ferrous ions to ferric ions (Ogawa et al., 2003). A 0.2-ml aliquot of the emulsion sample was added to 0.5 ml of isooctane/2-propanol (3:1 v/v) extraction solvent. The mixture was vortexed three times for 10 s each and centrifuged for 8 min at 720×g at room temperature, after which 50 μl of the separated organic phase was added to 2.95 ml of methanol/butanol (2:1 v/v) mixture. This was followed by the addition of 15 μl of 3.94 M ammonium thiocyanate aqueous solution and 15 μl of 0.072 M ferrous iron acid solution. The 3.03 ml aliquot was vortexed, incubated for 20 min at room temperature, then the absorbance at 510 nm was measured against a ferric ion standard curve. All absorbance measurements were corrected by an average of three blank measurements, in which the 50-μl volume of the separated organic phase was replaced by 50 μl of methanol/butanol (2:1 v/v) mixture.

A ferric ion standard curve was created based on the measurement of ferric ion dilutions of a 10 μg/ml stock solution. To make the ferric ion stock solution, 0.5 g of iron powder was dissolved in 50 ml of 10 N hydrochloric acid and 1-2 ml of 30% hydrogen peroxide was added. The mixture was boiled for 5 min to remove excess hydrogen peroxide, cooled, and then diluted to 500 ml with deionized water. A 1-ml aliquot was further diluted to 100 ml with methanol/butanol (2:1 v/v).

To make the ferrous iron acid solution, two reagents were prepared: (i) 2 g of ferrous sulfate heptahydrate was dissolved in 50 ml of deionized water; and (ii) 1.6 g of barium chloride dehydrate was dissolved in 50 ml of deionized water. The second reagent was slowly added to the first reagent, then 2 ml of 10 N hydrochloric acid was added. The mixture was centrifuged at a low speed to fully precipitate the sedimentating barium sulphate, and the clear supernatant was kept in the dark at 2° C. and used during the peroxide assay.

### 2.6 Emulsion Drop Size Measurement

Emulsion samples were diluted 100 fold with 0.02 M disodium phosphate buffer at the respective pH values. Each duplicate was measured twice for particle size (Z-average) at 25° C. by dynamic light scattering (DLS), using a Zetasizer Nano ZS90 with optical arrangement at 90° (Malvern Instruments Ltd., UK). Each measurement was comprised of 15 trial runs of 10 s each.

The average value of Z-average (Z_{avg}) was converted to the average sauter mean diameter (d₃₂) (Thomas, 1986) as:

Avgd₃₂=Z_{avg}×(1+Q)² (1)

where Q was the average experimentally determined polydispersity obtained during particle size measurement.

### 2.7 Calculations of Peroxide Value and Intrinsic Rate of Oxygen Depletion

The peroxide value (PV) of an emulsion sample was calculated as:

$$PV [ mmol peroxide formed / kg of lipid ] = \frac{\frac{N [ mol ]}{2 \times 50 [ μ l ] \times {10}^{- 3} ( ml / μ l )} \times \frac{0.5 [ ml ]}{0.2 [ ml ]} \times \frac{1}{0.02}}{0.93 [ g / ml ]} \times {10}^{3} [ g / kg ] \times {10}^{3} [ mmol / mol ] \qquad ( 1 )$$

where the factor “2” was to account for 1 hydroperoxide molecule reduced for every 2 ferric ions formed in the assay (Cayman Chemicals); 50 μl was the volume of the separated organic phase analyzed; 0.5 ml was the volume of extraction solvent; 0.2 ml was the volume of emulsion sample used; 0.02 was the volumetric fraction of dispersed phase of fish oil in emulsion; 0.93 was the density of the fish oil; and

$$N [ mol ] = \frac{Abs}{k [ ml / μg ]} \times {10}^{- 6} [ g / μg ] \times \frac{3.03 [ ml ]}{55.847 [ g / mol ]} \qquad ( 2 )$$

where N was the number of moles of ferric ion formed in 3.03 ml of aliquot; Abs was the average absorbance reading of a sample emulsion corrected by that of the blank; k was the slope from the ferric ion standard curve; and 55.847 g/mol was the formula weight of iron. The stoichiometric ratio of oxygen, hydroperoxide, and ferric ion is 1:1:2.

The total rate of oxygen depletion during oxidation of oil was proportional to the rate of peroxide formation:

$$\frac{\partial ( {Abs}_{t} - {Abs}_{0} )}{\partial t}$$

which was in turn proportional to (6/d₃₂)× (Intrinsic rate of oxygen depletion per unit of interfacial area of the emulsion drops),
where Abs₀ was the average absorbance reading of a sample emulsion corrected by that of the blank at time=0; Absₜ was that at time=t; and (6/d₃₂) represented the ratio of the total surface area of the emulsion drops to total volume of the drops.

Hence, the relative intrinsic rate of oxygen depletion per unit of interfacial area of the emulsion drops was calculated as:

$$\frac{\partial ( {Abs}_{t} - {Abs}_{0} )}{\partial t} \times \frac{{d}_{32}}{6} \qquad ( 3 )$$

### 2.8 Zeta Potential Measurement

Zeta potential of 0.5% (w/v) protein solutions was measured at 25° C., using a Zetasizer Nano ZS90. All data were collected in two measurements, each comprising 20 trial runs.

### 2.9 Turbidity Assay

Protein solutions at 0.5% (w/v) were read at 600 nm as a measurement of turbidity.

### 2.10 Circular Dichroism

Circular dichroism spectra were collected using a Jasco J-810 spectrometer (Jasco Spectroscopic Co.), for protein solutions diluted in 0.02 M sodium diphosphate without sodium azide. A quartz cuvette with a path length of 2 mm was used for 0.005% (w/v) protein solutions adjusted to pH 3 and 7, while that with a path length of 0.1 mm was used for 0.01% (w/v) solutions adjusted to pH 9 and 12.5. Ellipticity (mdeg) data were collected at 25° C. in continuous scanning mode in the wavelength of 190-260 nm, with the bandwidth set at 2 nm, data pitch at 0.2 nm, the response time at 4 s, and the scanning speed at 50 nm/min. The average spectrum for each sample was plotted using the Spectra Manager software from Jasco, based on at least two scans. Secondary structure was predicted from the deconvolution of the average spectrum by the online server Dichroweb (Whitmore and Wallace, 2004), using the reference data set 7 (Janes, 2008) and the CONTINLL algorithm (Provencher and Glockner, 1982; Van Stokkum et al., 1990).

### 2.11 TNBS Assay

The TNBS assay was used to determine the degree of hydrolysis (DH). The procedure was adapted from Adler-Nissen (1986). Protein solutions (0.5% w/v) protein were vortexed with 2% (w/v) SDS solution in a 1:1 (v/v) ratio. A 0.25-ml aliquot of the mixture was added to 2 ml of 0.2125 M sodium diphosphate buffer at pH 8. Next, 2 ml of 0.1% (w/v) TNBS in water was added. The entire mixture was vortexed and incubated in a 50° C. water bath in the dark for 1 h. After incubation, the reaction between TNBS and protein was inactivated by adding 4 ml of 0.1 N hydrochloric acid and immediate cooling to room temperature. After 30 min, absorbance was read at 340 nm, against a leucine (0-1.5 mM) standard curve. All data were collected in three measurements.

DH was calculated as:

$$DH ( \% ) = \frac{{h}_{eqv}}{{h}_{total}} \times 100 \qquad ( 4 )$$

where h_{eqv} was the amount of peptide bonds cleaved in equivalent (meqv/g of protein), and hₜₒₜₐₗ, estimated at 9.1, was the sum of number of amino acid residues in the heterotrimer molecules per gram of soy 7S.

### 2.12 ANS Assay

A 400-0, aliquot of 0.5% (w/v) protein solution was vortexed with 10 μL of 4.8×10⁻⁴ M ANS in ethanol and incubated in the dark for 1 h at room temperature. An 80-μL aliquot of the mixture was assayed in a 96-well black plate with flat bottom (Costar®) using a Flux Station II fluorescence spectrophotometer. The excitation wavelength was 360 nm, and emission fluorescence spectra in the wavelength range of 450 to 550 nm were collected. All data were collected at 25° C. in two measurements.

### 2.13 Detection of Intermolecular β-Sheet Fibrils at Oil/Water Interface with Thioflavin-T Dye

The thioflavin-T fluorescent dye was used to stain β-sheet fibrils. A 3.0 mM stock solution of the dye was freshly prepared by dissolving 9.6 mg of the dye in 10 ml of a pH 7 phosphate buffer (10 mM disodium phosphate, 0.15 M sodium chloride). The stock solution was diluted by a factor of 50 in the same buffer, and this working solution was used on the day of preparation (Kroes-Nijboer et al., 2009) for binding β-sheet fibrils in samples.

2.13.1 Confocal Laser Scanning Microscopy

An emulsion sample was centrifuged under at 5000×g for 15 min, and thereafter 24 μl of the cream was pipetted into 2 ml of thioflavin-T working solution (Section 2.13), vortexed, and examined under a Nikon MR confocal inverted microscope. The thioflavin-T dye was excited by a 440 nm laser line. Emissions were collected using a 60×(1.4 NA) oil objective with a 482/35 bandpass filter and 0.7 AU pinhole. The scan was line averaged (4×) and over-sampled for high resolution.

2.13.2 Fluorescence Spectrophotometric Assay

The detection of β-sheet fibrils by fluorescence spectrophotometry was described by Bolder et al. (2007b). The samples stained included emulsions and their respective constituting hydrolysate solutions. A 24-μl aliquot of each sample was pipetted into 2 ml of thioflavin-T working solution (Section 2.13). The mixture was vortexed and refrigerated at 2° C. for 12-15 h to allow sufficient time for staining. The fluorescence was measured using a Cary Eclipse fluorescence spectrophotometer, with the excitation wavelength set at 440 nm (slit width 10 nm) and the emission spectrum collected from 460 nm to 500 nm (slit width 10 nm). The slowest scan rate at 30 nm/min was selected. The fluorescence intensity of fibrils at the interface was obtained in duplicate assays, from the subtraction of the fluorescence intensity of an emulsion by that of its constituting hydrolysate solution.

### 2.14 Raman Spectroscopy of Hydrolysate Protein Adsorbed onto Flat Functional Substrates

In the present study, Raman spectroscopy was adapted to monitor the formation of protein intermolecular β-sheets on a functional silver substrate in a liquid environment. The silver layer was modified to represent the charge property of the oil, using modifying agents that deposit on the silver as self-assembled monolayers (SAM).

2.14.1 Preparation of Substrates

Microscopic glass slides were deposited with silver to form reflective surfaces. The silver surface was then functionally modified. Chamber wells were fixated on the functional silver surface. The chamber well was made by hole-punching a baking silicone sheet, cutting the template out, and sticking the template onto the glass slide using epoxy glue.

The method to create a silver surface was from the MRSEC interdisciplinary education group of University of Wisconsin Madison (http://mrsec.wisc.edu/edetc/nanolab/ab/agthiol/). First, an active silver solution was prepared in the following sequence: (1) Concentrated ammonium hydroxide was added dropwise to 2.5 ml of 0.1 M silver nitrate solution until the initial precipitate dissolved. (2) 1.25 ml of 0.8 M potassium hydroxide was added, resulting in the formation of a dark precipitate. (3) More concentrated ammonium hydroxide was added dropwise to re-dissolve the precipitate. In the dark, on a glass slide placed with an open petri dish, 8 drops of 0.5 M glucose were added, followed by the addition of 25 drops of the active silver solution. The petri dish was gently agitated and within few minutes a dark precipitate formed. The dark precipitate was rinsed off with distilled water to reveal the silver coating underneath. The glass slide was kept in the dark. The next day residual silver oxide on the silver coating was gently rubbed off in water using wetted Kimwipe paper. The silver coated glass slide was then air-dried. Silver-coated glass slides were made hydrophobic, by being stored for a day in screw-capped glass bottles in which a few drops of HMDS was added. HMDS would vaporize and become deposited onto the silver. To make a silver-coated glass slide anionic, 1 mM of MES was added dropwise to cover the silver coating for 10 min, after which the MES solution was drained off and the glass slide was air-dried. This would leave the silver layer functionalized with negative sulfite headgroups (Coronado et al., 2005).

2.14.2 Acquisition of Raman Spectra

A Senterra Raman confocal microscope from Bruker Optics was used. A 633-nm laser line at 20 mW power and a 50×objective lens (0.50 NA) were used to acquire signals of protein solutions (in 0.02% sodium azide, 0.02 M disodium phosphate) at 2% (w/v) or otherwise stated, at a 3-5 cm⁻¹ spectral resolution, within a frequency range of 400-1800 cm⁻¹ wavenumber. The aperture was set at 2 mm, and the integration time for each scan was 10 s. To study the adsorption behaviour of protein onto a functional substrate, less than 50 μl of a protein solution sample was deposited into the chamber well. Three scans were collected at three different scan points at the focal plane of the silver, immediately after deposition and on the next day. The solution sample was prevented from drying in between the days.

Raman spectra of buffer solutions and distilled water were collected at the focal plane of the silver as blanks. β-lactoglobulin (2% w/v in pH 7 distilled water) was used as a positive control for the signal of the Amide I range, which signified the presence of protein. Soy 7S hydrolysate from acid hydrolysis (2% w/v in 0.02 M disodium phosphate pH 3 buffer, ˜2.6 DH %) served as a model protein that was rich in intermolecular β-sheet, due to the presence of β-sheet fibrils. The OPUS 6.5 software was used for spectral processing, during which a frequency range with a linear baseline (˜1200-1800 cm⁻¹) was cut out, subjected to baseline correction under interactive mode (using a 64-baseline-point rubberband correction method), and then treated by a 25-point smoothing.

### 3. RESULTS

### 3.1 Degree of Hydrolysis Attainable as a Function of Hydrolytic Conditions

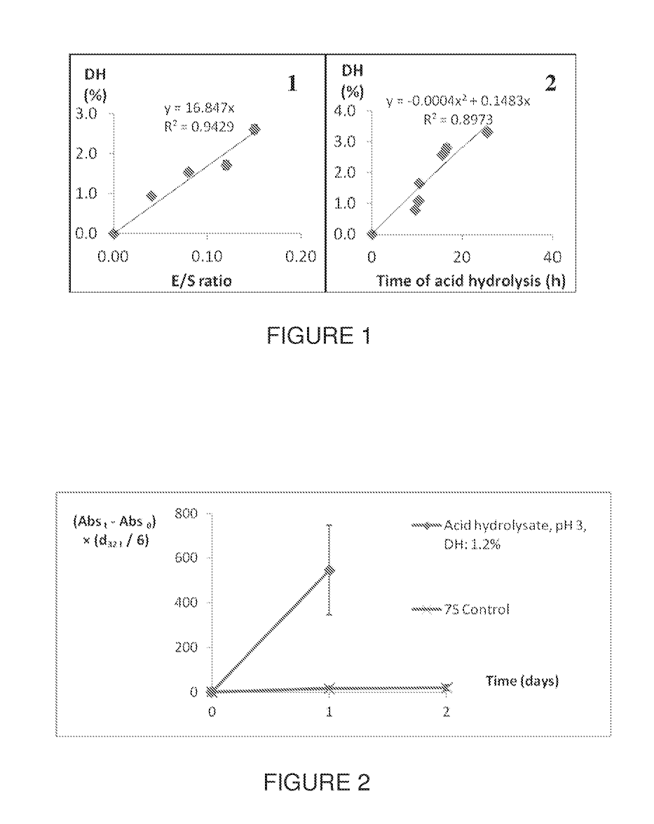

FIG. 1 shows the mathematical equations of trendlines that describe the change in DH of soy 7S as a function of either the E/S ratio during trypsinization, or the time of acid hydrolysis. The equations were used to estimate and verify the DH that was attained during the preparation of 7SH samples. Even at a high E/S ratio of 0.15 and a time as long as 25.5 h during acid hydrolysis, the DH could not reach 4%, probably as a result of the large and compact native state of the 7S (the isolation procedure did not involve any heat treatment).

### 3.2 Effect of Method and Degree of Hydrolysis

A gel-like appearance of 7SH from acid hydrolysis, which got more turbid with increasing incubation time, in contrast to the native 7S solution and 7SH from trypsinization which were generally clear. Table 1 shows that even though the proportion of unordered secondary structure increased expectedly with acid hydrolysis, suggesting increased molecular flexibility, the emulsions stabilized by the obtained 7SH were physically less stable than those stabilized by the native 7S control in terms of phase separation. The oxidative stability was also poorer, as could be seen in FIG. 2.

TABLE 1
Characterization of soy 7S hydrolysates with different degrees of hydrolysis
(DH) from acid hydrolysis in terms of secondary structure, and the physical stability
of the emulsions they formed at 0.5% (w/v) protein concentration in 0.02M disodium
phosphate buffer. (1) 10.25 h at pH 3; (2) 15.5 h at pH 3; (3) 25.5 h at pH 3;
(4) 10.25 h at pH 7; (5) 15.5 h at pH 7; (6) 25.5 h at pH 7

| | pH | | | | | | | |
|---|---|---|---|---|---|---|---|---|
| | 3 | | | | 7 | | | |
| | Sample | | | | | | | |
| | Control 7S | A | B | C | Control 7S | D | E | F |
| DH (%) | 0 | 1.5 | 2.2 | 3.5 | 0 | 1.5 | 2.2 | 3.5 |
| Secondary structure: | | | | | | | | |
| Random coil | 0.413 | 0.417 | 0.471 | 0.506 | 0.410 | 0.458 | 0.435 | 0.456 |
| α-helix | 0.161 | 0.139 | 0.166 | 0.080 | 0.135 | 0.114 | 0.096 | 0.077 |
| β-sheet | 0.426 | 0.444 | 0.363 | 0.414 | 0.456 | 0.428 | 0.469 | 0.467 |
| Emulsion physical stability: | | | | | | | | |
| Day emulsion broke | 1 | 4 | 4 | 0 | 4 or Nil | 0 | 0 | 0 |

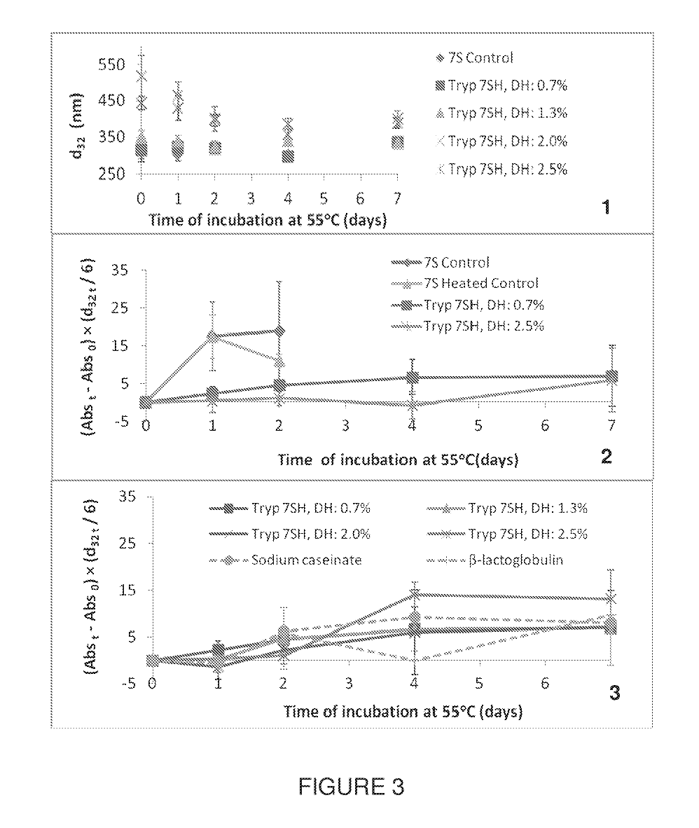

FIG. 3, Panel 2 delineates that 7SH from trypsinization yielded significantly better oxidative stability than the native 7S control and the heated 7S control. The heated 7S control was tested to verify that the enhanced oxidative stability was the effect of trypsinization, and not due solely to the heat treatment carried out during trypsin inactivation. The decrease in the initial rate of oxidation became more evident at a higher DH of 2.5% than 0.7%. However, at the range of low DH (0.7-2.5%), the surface hydrophobicity and composition of protein secondary structure were generally very similar (Table 2), as was the oxidative stability of their corresponding emulsions (FIG. 3, Panel 3). Such low DH values made the oxidative stability comparable to that of milk proteins sodium caseinate and P-lactoglobulin at pH 7 and the same conditions of emulsification. Interestingly, though, FIG. 3, Panel 1 shows that DH influenced the emulsion drop size, with the initial d₃₂ being larger as DH increased and this increase being more pronounced at DH of 2.0% and 2.5%. The d₃₂ of emulsions stabilized by 7SH were relatively constant throughout the seven days of incubation at 55′C with the variation being within the experimental error.

TABLE 2
Characterization of soy 7S hydrolysates with different degress of hydrolysis (DH) from
trypsinization, in terms of secondary structure and surface hydrophobicity as indicated
from the ANS assay

| | pH | | | | |
|---|---|---|---|---|---|
| | 7 | | | | |
| DH (%) | 0 | 0.7 | 1.3 | 2 | 2.5 |
| Secondary structure: | | | | | |
| Random coil | 0.410 | 0.460 | 0.471 | 0.471 | 0.452 |
| α-helix | 0.135 | 0.102 | 0.102 | 0.096 | 0.084 |
| β-sheet | 0.456 | 0.439 | 0.428 | 0.433 | 0.464 |
| ANS fluorescence intensity | 24062 | 37352 | 38946 | 34704 | 33241 |
| at λ: 460 nm (A.U.) | (±907 SD) | (±755 SD) | (±1302 SD) | (±1323 SD) | (±576 SD) |

### 3.3 Effect of Protein Concentration and Ionic Strength on Hydrolysates from Trypsinization

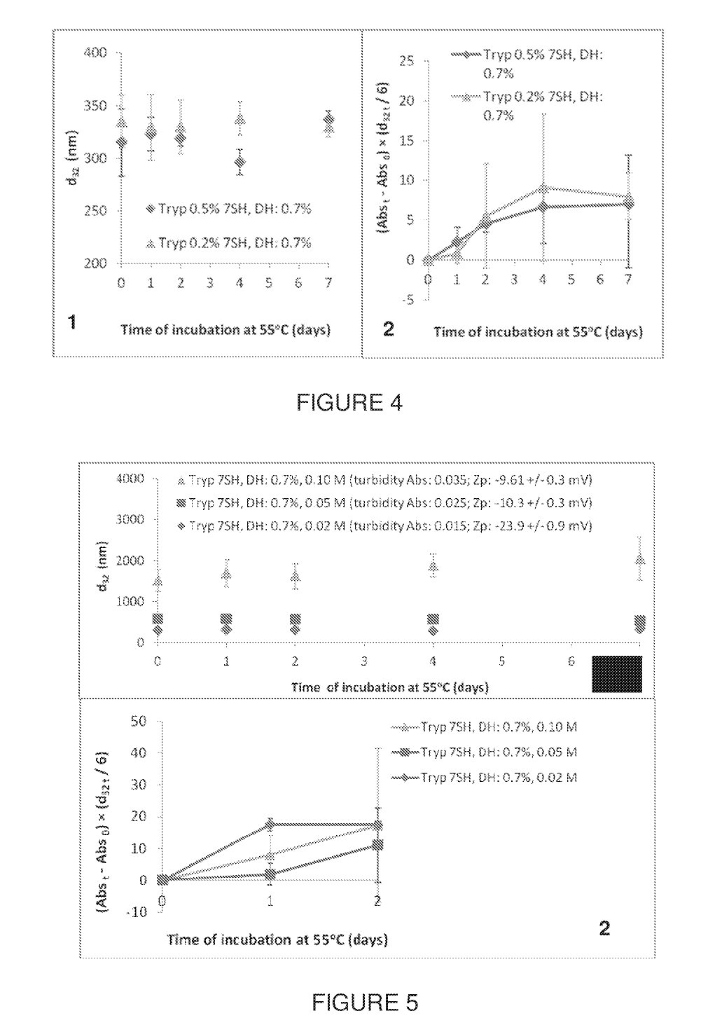

FIG. 4, Panels A and B show that at pH 7 and an ionic strength of 0.02 M 20 disodium phosphate, the physical and oxidative stability of a 2% (v/v)-oil emulsion stabilized by a 0.2% (w/v) concentration of 7SH was not significantly different from that stabilized by a 0.5% (w/v) 7SH. However, the results in FIG. 5, Panel 2 suggested that the ionic strength had a significant influence on the initial intrinsic rate of oil oxidation. The overall peroxide value results as shown in the insert of FIG. 5, Panel 2 did not differ significantly with ionic strength. Therefore, the difference in intrinsic rate of oxidation (rate per unit interfacial area) can be attributed to a decrease in the surface area per unit volume of emulsion drop (inverse of d₃₂) with ionic strength, as can be seen from FIG. 5, Panel 1.

### 3.4. Effect of pH and Surface Charge of Oil on Hydrolysates from Trypsinization

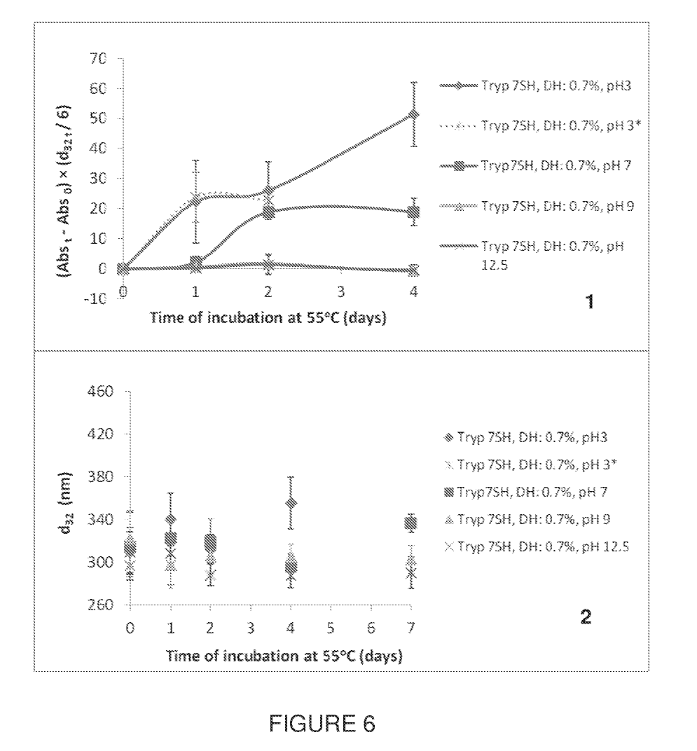

The pH value was found to be a very important factor determining the oxidative stability of the emulsion, albeit it did not seem to influence the emulsion drop size as depicted in FIG. 6, Panel 1. FIG. 6, Panel 2 shows that oxidative stability was the poorest at pH 3, regardless of whether the oil surface was non-polar or made anionic by the coverage of DATEM (in our initial experiments it was found that 4% Menhaden oil emulsions stabilized only by 0.28% DATEM had a very high average zeta potential of −15 88 mV at pH 3). FIG. 6, Panel 2 also illustrates that as the pH became more alkaline after pH 7, the initial intrinsic oxidation of the emulsion was effectively more retarded. The trend shown in FIG. 6, Panel 2 might be related to the physical characteristics of the 7SH protein as detailed in Table 3.

TABLE 3
Characterization of 7SH of 0.7% degree of hydrolysis (DH) from
trypsinization, at different pH values, in terms of secondary structure,
zeta potential, surface hydrophobicity as indicated from the ANS asssay,
and the conjectured protein structure based on the aforementioned properties

| | pH | | | | |
|---|---|---|---|---|---|
| | Control 7S (pH 7) | 3 | 7 | 9 | 12.5 |
| Secondary structure: | | | | | |
| Random coil | 0.410 | 0.446 | 0.471 | 0.549 | 0.625 |
| α-helix | 0.135 | 0.126 | 0.102 | 0.163 | 0.140 |
| β-sheet | 0.456 | 0.430 | 0.428 | 0.288 | 0.135 |
| Zeta potential (mV) | −25.6 | 10.7 | −23.9 | −31.5 | −17.6 |
| | (±0.1 SD) | (±0.1 SD) | (±0.9 SD) | (±0.8 SD) | (±0.2 SD) |
| ANS fluorescence | 24062 | 106658 | 37352 | 45139 | No peak λ |
| intensity at λ: 460 nm | (±907 SD) | (±295 SD) | (±755 SD) | (±106 SD) | |
| (A.U.) | | | | | |
| Conjectured protein | Intact | Loose, like | Slightly loose | Unfolded | Extensively |
| structure | | molten globule | | | unfolded |

With the increase in pH from 3 to 12.5, the proportion of unordered protein secondary structure increased from 44.6% to 62.5%, while the proportion of β-sheet decreased from 43.0% to a mere 13.5%. The zeta potential decreased from 10.7 mV at pH 3 to −31.5 mV at pH 9, while it increased to −17.6 mV at pH 12.5. Both signs suggested an increase in opening up and flexibility of the protein molecule, until it became extensively unfolded with many buried hydrophobic amino acids exposed by pH 12.5. The decrease in ANS fluorescence intensity at the peak emission wavelength of 460 nm, with the increase of pH from 3 to 7-9, also inferred the gradual loss of a local hydrophobic environment for the binding ANS dye due to protein unfolding. At pH 12.5, it was postulated that the protein was so extensively unfolded that such a local environment was lost, and thus there was no binding of ANS and no fluorescence peak detected at 460 nm. It was unlikely that the lack of an ANS fluorescence peak at pH 12.5 was due to instability of ANS at such an alkaline pH, since the dye had been reportedly used to probe protein structure at a pH value as high as 13 (Sen et al., 2008). Therefore, at pH 3, the protein retained its secondary structure though fluorescence intensity of bound ANS increased significantly thereby indicating a loose tertiary structure, possibly indicating a structure similar to molten globule state. As pH increased, however, the protein tended to lose its secondary structure.

The emulsions created by high pressure homogenization at 20 kpsi generally had a proteinaceous interfacial layer laden with β-sheet fibrils, as inferred from the clear staining at the rims of the emulsion drops by the thioflavin-T dye in FIG. 8. The results of the quantification of β-sheet fibrils found in the continuous phase and interfacial layer of emulsions were shown in Table 4.

TABLE 4
Determination of the thioflavin-T dye intensity at the interfacial
layer of emulsions stabilized by 7SH and the native control

| | | | Thioflavin-T fluorescence intensity | | |
|---|---|---|---|---|---|
| Conditions | | | Emulsion | Aqueous phase | Interface |
| Control, 0.02M, pH 7 | | | 41.0 (±5.7 SD) | 13.0 (±0.0 SD) | 28.0 (±5.7 SD) |
| 7SH (tryp) | 0.02M | pH 3 | 43.0 (±5.7 SD) | 15.0(±1.4 SD) | 28.0 (±5.8 SD) |
| DH: 0.7 | | pH 3, with 0.2% Datem | 67.5 (±2.1 SD) | 15.0 (±1.4 SD) | 52.5 (±2.5 SD) |
| | | pH 7 | 39.0 (±2.8 SD) | 13.5 (±0.7 SD) | 25.5 (±2.9 SD) |
| | | pH 9 | 37.5 (±2.1 SD) | 13.0 (±1.4 SD) | 24.5 (±2.5 SD) |
| | | pH 12.5 | 41.5 (±7.8 SD) | 10.0 (±2.8 SD) | 31.5 (±8.3 SD) |
| | 0.05M | pH 7 | 38.0 (±1.4 SD) | 15.0 (±0.0 SD) | 23.0 (±1.4 SD) |
| | 0.10M | pH 7 | 39.5 (±4.9 SD) | 17.0 (±0.0 SD) | 22.5 (±4.9 SD) |
| 7SH (acid) | 0.02M | pH 3 | 79.0 (±7.1 SD) | 82.0 (±2.8 SD) | −3.0 (±7.6 SD) |
| DH: 1.2 | | | | | |

Three main points were observed. First, the 7SH from acid hydrolysis, which led to the poorest oxidative stability of all the emulsions tested, had the highest amount of β-sheet fibrils in the continuous phase, but negligible amount at the interface. Second, although the native 7S and the 7SH from trypsinization at varying pH values conferred different oxidative stability to their corresponding emulsions, the amount of β-sheet fibrils in the interfacial layer was similar. Third, when the oil was first stabilized by DATEM followed by a second layer of cationic 7SH from trypsinization at pH 3, a near two-fold increase in β-sheet fibrils was induced, compared to the case without DATEM at pH 3.

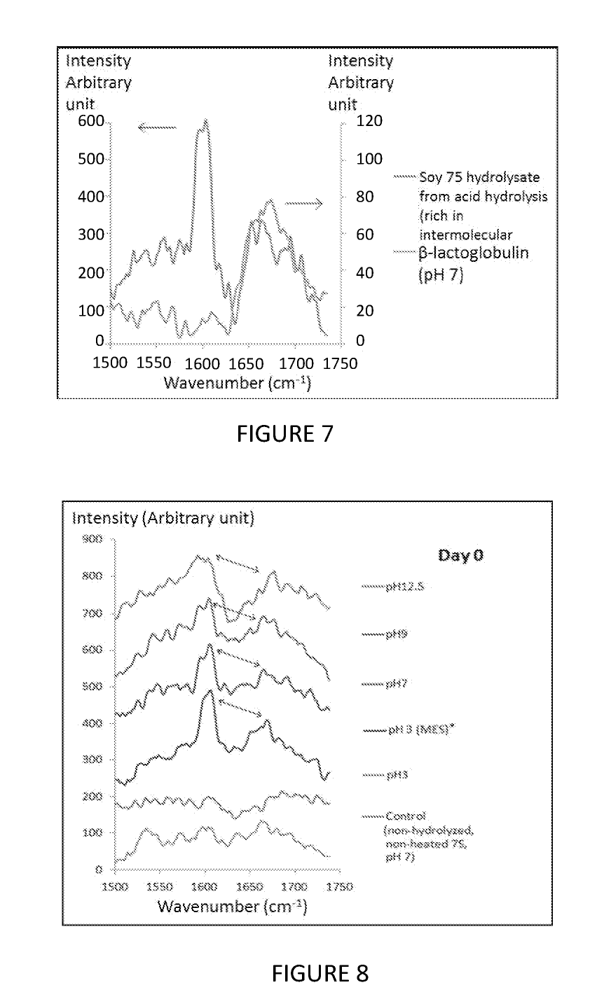

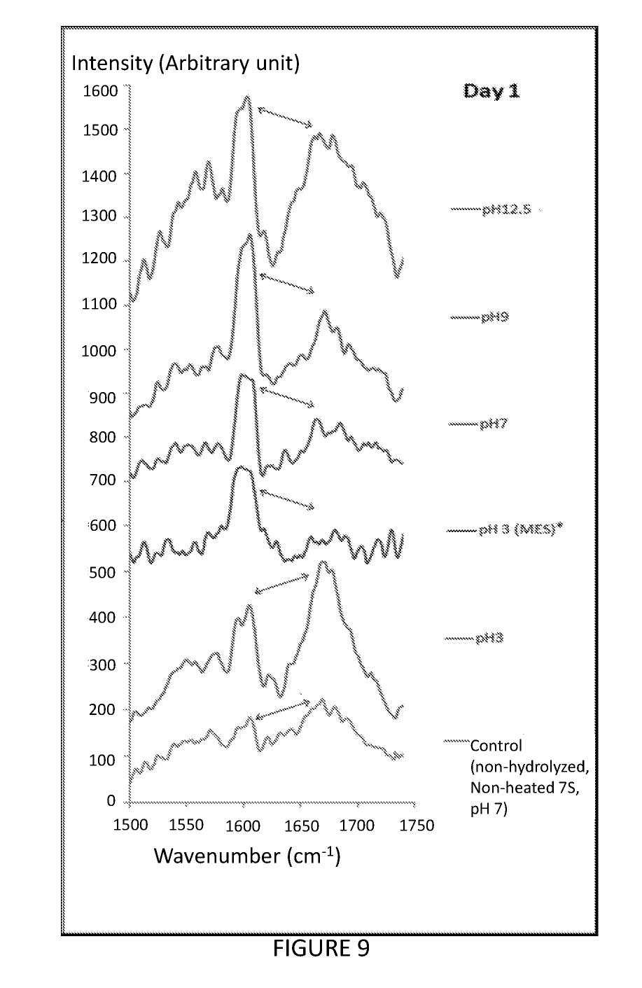

Despite the lack of evident difference in the thioflavin-T fluorescence results of the native 7S and trypsinized 7S at varying pH values, the Raman spectra in FIGS. 8 and 9 show otherwise. For trypsinized 7S at pH 7, 9 and 12.5, there was formation of intermolecular β-sheets (with a characteristic peak at ˜1600 cm⁻¹ as verified in FIG. 7) on the hydrophobic silver surface immediately after deposition as depicted in FIG. 8. The ˜1600 cm⁻¹ band observed was very close to 1604 cm⁻¹, which had been assigned to intermolecular β-sheets by other authors using the complementary technique infrared spectroscopy (Sharma et al., 1999; Villar-Piqué et al., 2010). The pH values 7, 9, and 12.5 were associated with better emulsion oxidative stability compared to the native 7S at pH 7 and trypsinized 7S at pH 3. FIG. 9 depicts that one day after depositing the protein solutions, the signals for intermolecular β-sheet structure became stronger with the increase in pH from 7 to 12.5. In contrast, for the native 7S and the trypsinized 7S at pH 3, the signals at ˜1600 cm⁻¹ were never stronger than the signal in the Amide I range typical of protein (with a peak within 1650-1670 cm⁻¹) as confirmed by similar observations (not shown) at lower protein concentrations. FIG. 8 revealed that intermolecular β-sheet structure was induced on the silver surface made anionic by the MES chemical, soon after the deposition of trypsinized 7S at pH 3. However, instead of a big increase in signal at ˜1600 cm⁻¹ on the next day, as would have been expected based on the strong thioflavin-T fluorescence seen in Table 4, the intensity of its ˜1600 cm⁻¹ peak in FIG. 9 was weaker than that for the trypsinized 7S adsorbed on the hydrophobic silver surface at pH 9 and 12.5.

### 5. DISCUSSION

The thioflavin-T dye is known to be specific for the structural motif in β-sheet fibrils by binding such that its axis lies parallel to the length of the fibril (Khurana et al., 2005; Krebs et al., 2005). The very high intensity of thioflavin-T fluorescence in 7SH from acid hydrolysis, on top of the gel-like appearance, suggested that the formation of β-sheet fibrils was strongly induced as a result of acid hydrolysis. However, fibrillogenesis in the continuous aqueous phase, apparently, adversely affected the amount of mobile protein that could become adsorbed onto the oil interface during emulsification, as evident from Table 4. It was clear that the fibrils formed exhibited even poorer emulsifying properties than the native 7S control, resulting in lower emulsion stability, both physically and oxidatively. Sagis et al. (2008) and Humblet-Hua et al. (2011) reported that protein fibril-reinforced capsule shells, formed by the layer-by-layer polyelectrolyte deposition technique with other polymers of opposite charge, such as pectin, possessed high tunable mechanical strength. However, the results in the present study suggested that pre-formed protein fibrils alone were inadequate to control oxygen permeability across the emulsion interface.

Nonetheless, FIG. 3, Panel 2 proved that hydrolysis of 7S, when achieved by trypsinization, was useful in the improvement of emulsion oxidative stability Limited trypsinization within 0.7-2.5% DH impeded the rate of oil oxidation to rates comparable with emulsions stabilized by sodium caseinate and P-lactoglobulin, at the same conditions of emulsification. Note-worthily, at pH 7 and a low ionic strength of 0.04 M Nat, the milk proteins conferred similar emulsion oxidative stability. The ability of trypsinized 7SH to provide oxidative stability was however compromised at higher ionic strengths. This may be because of the tendency of protein to form clusters more easily as a result of reduced electrostatic intermolecular interactions (as evidenced by reduced zeta potential). The adsorption of such clusters onto drop interface may lead to a looser packing of proteins in the interfacial layer. In addition to the acid hydrolysis of proteins at a high temperature, other researchers showed that β-sheet fibrils were also detected by Thioflavin-T in heat induced aggregates of specific proteins naturally rich in β-sheet structure, such as P-lactoglobulin and ovalbumin (Carrotta et al., 2001; Azakami et al., 2005; Stirpe et al., 2008). Interestingly, Table 4 indicates that there was 3-sheet fibrillar structure induced at the proteinaceous interface of emulsions stabilized by the native 7S and the trypsinized 7S. To our knowledge, this was the first report of a new circumstance in which fibrillar features were induced and detected by Thioflavin-T at the interface. The limit in resolution of confocal scanning laser microscopy restricted the detection of the spatial arrangement of the fibrils induced at the interface. The results displayed in Table 4 were also only relative quantification of the β-sheet fibrils in total. However, the results from Raman spectroscopy allowed further insight, as they were indicative of protein secondary and supersecondary structure at the immediate contact surface of the silver, which was modified functionally to mimick the oil surface. This was considered a result of combined effect of different phenomena: (1) possible surface-enhanced Raman scattering (SERS) effect due to the presence of the silver layer; (2) the optical slice capability of the Raman confocal microscope; and (3) the reduced scattered light intensity with increasing distance from the focal point, in an inverse-square relation. From FIGS. 8 and 9, it was inferred that compared to the native 7S, trypsinized 7S at pH 7, 9 and 12.5 could readily form intermolecular β-sheet fibrils that were aligned closely to the hydrophobic surface, and this was especially apparent at pH 9 and 12.5 one day later. In contrast, at pH 3, fewer intermolecular β-sheet fibrils were formed in proportion to the total protein adsorbed after one day. Interestingly, this trend in intermolecular β-sheet formation on the hydrophobic silver surface matched the trend in the oxidation rate found in trypsinized 7S-stabilized emulsions, as depicted in FIG. 6, Panel 2.

It is well reported in literature that a stable molten globule state can be induced in many proteins at an acidic pH (Ikeguchi et al., 1986; Kuwajima, 1989; Sugawara et al., 1991; Fink et al., 1993; Mohsen Asghari et al., 2004; Naseem et al., 2004). Considering the limited hydrolysis attained in this study, it was postulated that trypsinized 7S at pH 3 existed as a molten globule with the hydrophobic amino acids still essentially tucked within a loose tertiary structure, and thus the great enhancement in ANS fluorescence upon binding that was reported in Table 3 (Collini et al., 2000). According to Wierenga et al. (2006), protein unfolding at an interface would only occur if the associated kinetics were similar or faster than the kinetics of adsorption. However, considering the high adsorption rate during HPH due to a high orthokinetic collision rate in a highly turbulent flow (Hakansson et al., 2009), it was conceivable that trypsinized 7S at pH 3 did not unfold as extensively at the oil surface as at alkaline pH (see Table 3). The inability to form adequate intermolecular β-sheet structure planar to the oil surface, as suggested in FIG. 9, might also infer the lack of an effective primary layer of protection and consequentially a high rate of oil oxidation.

The DATEM emulsifier was used in the study to design an anionic oil surface, with the intent of creating electrostatic attraction between the oil and the cationic trypsinized 7S at pH 3 so as to induce the formation of more β-sheet fibrils, and in turn hopefully improve the oxygen barrier property. The rationale behind this was supported by works of other authors. Lopes et al., (2007) reported that at a physiological pH, an anionic lipid surface could associate strongly with random-coiled islet amyloid polypeptide molecules, thereby driving a conformation transition to an alpha-helical and ultimately β-sheet fibrillar structure. Separately, Chi et al. (2008) reported that an anionic lipid membrane could induce the arrangement of Alzheimer's amyloid-β peptide molecules into β-sheet fibrils. In the present study, although the total amount of fibrils formed in interfacial trypsinized 7S at pH 3 almost doubled, as a result of the use of DATEM in oil emulsions, there was no parallel increase in the amount of intermolecular β-sheet detected on the MES-modified silver surface one day after deposition. The corresponding emulsion oxidative stability at pH 3 was also not improved. These findings suggested that, the quantity of β-sheet fibrils formed at the interfacial layer alone was not a determinant factor of oxidative stability. The lack of improvement in oxidative stability could be because an electrostatic attractive surface might not speed up unfolding of the molten globule at pH 3 after all, as suggested by Adams et al. (2002). In order words, hydrophobic interaction between the protein and the oil might be a more important criterion. In addition, the orientation of the fibrils at the interface might also play a part.

It was unexpected that 7SH from trypsinization, at pH 7 and especially pH 9 and 12.5, could form fibrils on the oil surface. Although no irreversible denaturation was induced at the alkaline pH values, since no precipitation was observed in the protein solutions, the ability of these hydrolysates to self-assemble despite the high electrostatic repulsive forces was still surprising. On the other hand, the conversion of protein into an unfolded, highly flexible state had been reported to be essential for subsequent fibril formation to occur (Goers et al., 2002). This was seemingly relevant to the behavior of trypsinized 7S on a hydrophobic surface at alkaline pH values reported in the current study. According to Dong et al. (1998), the likely pathway in the formation of fibrillar structure might have involved the formation of a transient nonnative alpha-helix, which was then readily converted to an intermolecular β-sheet structure. An interesting study was carried out by Kowalewski and Holtzman (1999), on the difference in the arrangement of Alzheimer's amyloid-13 peptide molecules on hydrophobic graphite and mica, which bore a slight negative charge. They discovered that, with the same protein concentration used in both cases, the height of the amyloid-β fibrils formed on the graphite was barely 1 nm, whereas that on mica was 5-6 nm. This may indicate that fibrils predominantly tend to orient parallel to the surface only on the more hydrophobic graphite and not on the more hydrophilic (charged) mica. This had a strong implication on the present work: that, whether the β-sheet fibrils formed in the interfacial layer were more orthogonal or parallel to the oil surface, depended on the charge at the oil surface itself. If there was any bearing on how well the encapsulated oil was protected from oxidation, it seemed that a parallel alignment of the fibrils would favor oxidative stability. All in all, it was postulated that with the increase in pH above pH 7, there was more close-knitted alignment of extended β-sheets assembled flat to the oil surface, which reinforced the protection of the encapsulated oil against oxidation in addition to protein unfolding that promoted protein-oil hydrophobic interaction. Perhaps, as Schladitz et al. (1999) conjectured, the parallel β-sheets closest to the oil surface then interconnected with β-sheets of other spatial alignment with the increase in interfacial thickness. In any case, it was an interesting finding to learn of the capacity of trypsinized 7S to form amyloid β-sheet fibrils on a hydrophobic surface, since not all proteins with native principal β-sheet content develop into amyloid fibrils (Sambasivam et al., 2008). On the other hand, the improved emulsion oxidative stability was deemed not to be a result of increased thiol-disulfide interchange reactions and new disulfide bridge formation with increased unfolding, since soy 7S is poor in sulfur-containing amino acids (Shortwell and Larkins, 1989).

### 6. CONCLUSION

Hydrolysis of 7S via trypsinization improved emulsion oxidative stability in the pH range of 7 to 12.5 at low ionic strength Acid hydrolysis of 7S, however, resulted in poor physical and oxidative emulsion stability. Based on Raman spectroscopy of adsorbed 7SH, it is suggested that the orientation of the β-sheet fibril-assembly parallel to the oil interface tends to provide better oxidative stability.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicate herein or otherwise clearly contradicted by context. The use of any and all examples, or exemplary language (e.g., “such as”) provided herein, is intended merely to better illuminate the invention and does not pose a limitation on the scope of the invention unless otherwise claimed. The compositions and methods described herein can consist, consist essentially, or comprise the ingredients, steps and/or other characteristics identified. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. In addition, all publications cited herein are indicative of the abilities of those of ordinary skill in the art and are hereby incorporated by reference in their entirety as if individually incorporated by reference and fully set forth.

### REFERENCES

The following references, some of which are cited hereinabove, are indicative of the abilities possessed by those of ordinary skill in the art to which the invention pertains, and are hereby incorporated by reference in their entirety.
Adams, S., Higgins, A. M., and Jones, R. A. L., 2002, “Surface-mediated folding and misfolding of proteins at lipid/water interfaces,” Langmuir, Vol. 18, No. 12, pp. 4854-4861.
Adler-Nissen, J., 1986, Enzymic Hydrolysis of Food Proteins, Elsevier Applied Science Publishers, London, p. 427.
Akkermans, C., van der Goot, A. J., Venema, P., Gruppen, H., Vereijken, J. M., van der Linden, E., and Boom, R. M., 2007, “Micrometer-sized fibrillar protein aggregates from soy glycinin and soy protein isolate,” Journal ofAgricultural and Food Cehmistry, Vol. 55, pp. 9877-9882.
Badley, R. A., Atkinson, D., Hauser, H., Oldani, D., Green, J. P., and Stubbs, J. M., 1975, “The structure, physical and chemical properties of the soy bean protein glycinin,” Biochimica et Biophysica Acta, Vol. 412, pp. 214-228.
Barrett, A. J., Rawlings, N. D., and Woessner, J. F., 2004, Handbook or Proteolytic Enzymes, 2^{nd} edition, Elsevier, London.
Beermann, C., Euler, M., Herzberg, J., and Stahl, B., 2009, “Anti-oxidative capacity of enzymatically released peptides from soybean protein isolate,” European Food Research and Technology, Vol. 229, pp. 637-644.
Biswas, B. and Haydon, D. A., 1962, “The coalescence of droplets stabilised by viscoelastic adsorbed films,” Kolloid-Z, Vol. 185, pp. 31-38.
Bolder, S. G., Hendrickx, H., Sagis, L. M. C., and van der Linden, E., 2006, “Fibril assemblies in aqueous whey protein mixtures,” Journal of Agricultural and Food Chemistry, Vol. 54, pp. 4229-4234.
Bolder, S. G., Vasbinder, A. J., Sagis, L. M. C., and van der Linden, E., 2007a, “Heat-induced whey protein isolate fibrils: Conversion, hydrolysis, and disulphide bond formation,” International Dairy Journal, Vol. 17, pp. 846-853.
Bolder, S. G., Sagis, L. M. C., Venema, P., and van der Linden, E., 2007b, “Thioflavin T and birefringence assays to determine the conversion of proteins into fibrils,” Langmuir, Vol. 23, pp. 4144-4147.
Cayman Chemicals, “Lipid Hydroperoxide (LPO) Assay Kit Catalog No. 705002.” <www.progeninc.co.kr/pdf/LP0%20Assay %20Kit.pdf>, accessed May 2011.
Chen, H.-M., Muramoto, K., Yamauchi, F., Fujimoto, K., and Nohimara, K., 1998, “Antioxidant properties of histidine-containing peptides designed from peptide fragments found in the digests of a soybean protein,” Journal of Agricultural and Food Chemistry, Vol. 46, pp. 49-53.
Chi, E. Y., Ege, C., Winans, A., Majewski, J., Wu, G., Kjaer, K, and Lee, K. Y. C., 2008, “Lipid membrane templates the ordering and induces the fibrillogenesis of Alzheimer's disease amyloid-β peptide,” Proteins: Structure, Function, and Bioinformatics, Vol. 72: pp. 1-24.
Collini, M., D'Alfonso, L., and Baldini, G., 2000, “New insight on β-lactoglobulin binding sites by 1-anilinonaphthalene-8-sulfonate fluorescence decay,” Protein Science, Vol. 9, pp. 1968-1974.
Coronado, E., Forment-Aliaga, A., Romero, F. M., Corradini, V., Biagi, R., de Renzi, V., Gambardella, A., and del Pennino, U., 2005, “Isolated Mn₁₂ single-molecule magnets grafted on gold surfaces via electrostatic interactions,” Inorganic Chemistry, Vol. 44, pp. 7693-7695.
Damodaran, S., 1996, “Amino acids, peptides, and proteins,” In: Food Chemistry, 3rd edition, ed. Fennema, O. R. Marcel Dekker, Inc., p. 328.
deWit, I. N., Klarenbeek, G., and Swinkels, G. A. M., 1976, “The emulsifying properties of whey protein concentrates,” Zuivelzicht, Vol. 68, pp. 442-445.
Deru\tilde{e}re, J., Römer, S., d'Harlingue, A., Backhaus, R A., Kuntz, M., and Camara, B., 1994, “Fibril assembly and carotenoid overaccumuulation in chromoplasts: A model for supramolecular lipoprotein structures,” The Plant Cell, Vol. 6, pp. 119-133.
Dong, A., Matsuura, J., Manning, M. C., and Carpenter, J. F., 1998, “Intermolecular beta-sheet results from trifluoroethanol-induced nonnative alpha-helical structure in beta-sheet predominant proteins: infrared and circular dichroism spectroscopic study,” Archives of Biochemistry and Biophysics, Vol. 355, No. 2, pp. 275-281.
Goers, J., Permyakov, S. E., Permyakov, E. A., Uversky, V. N., and Fink, A. L., 2002, “Conformational prerequisites for α-lactalbumin fibrillation,” Biochemistry, 41: 12546-12551.
Howard, P. A., Lehnhardt, W. F., and Orthoefer, F. T., 1983, “7S and 11S vegetable protein fractionation and isolation,” U.S. Pat. No. 4,131,607, filed Aug. 10, 1981, and issued Jan. 11, 1983.
Humblet-Hua, K. N. P., Scheltens, G., van der Linden, E., and Sagis, L. M. C., 2011, Encapsulation systems based on ovalbumin fibrils and high methoxyl pectin. Food Hydrocolloids, Vol. 25, pp. 307-314.
Håkansson, A., Trägårdh, C., and Bergenståhl, B., 2009, “Studying the effects of adsorption, recoalescence and fragmentation in a high pressure homogenizer using a dynamic simulation model,” Food Hydrocolloids, Vol. 23, pp. 1177-1183.
Ikeguchi, M., Kuwaijima, K., Mitaru, M., and Sugai, S., 1986, “Evidence for identity between the equilibrium unfolding intermediate and a transient folding intermediate: A comparative study of the folding reactions of α-lactalbumin and lysozyme,” Biochemistry, Vol. 25, pp. 6965-6972.
Janes, R. W., 2008, “Reference datasets circular dichroism and synchrotron radiation circular dichroism spectroscopy of Proteins,” In: Modern Techniques in Circular Dichroism and Synchrotron Radiation Circular Dichroism Spectroscopy, eds. Wallace, B. A. and Janes, R. W. IOS Press.
Khurana, R., Coleman, C., Ionescu-Zanetti, C., Carter, S. A., Krishna, V., Grover, R. K., Roy, R., and Singh, S., 2005, “Mechanism of thioflavin T binding to amyloid fibrils,” Journal of Structural Biology, Vol. 151, pp. 229-238.
Koshiyama, I., 1968, “Chemical and physical properties of a 7S protein in soybean globulins,” Cereal Chemistry, Vol. 45, pp. 394-404.
Kowalewski, T. and Holtzman, D. M., 1999, “In situ atomic force microscopy study of Alzheimer's β-amyloid peptide on different substrates: New insights into mechanism of β-sheet formation,” Proceedings of the National Academy of Sciences, Vol. 96, pp. 3688-3693.
Krebs, M. R. H., Bromley, E. H. C., and Donald, A. M., 2005, “The binding of thioflavin-T to amyloid fibrils: localisation and implications,” Journal of Structural Biology, Vol. 149, pp. 30-37.
Krebs, M. R. H., Devlin, G. L., and Donald, A. M., 2009, “Amyloid fibril-like structure underlies the aggregate structure across the pH range for β-lactoglobulin,” Biophysical Journal, Vol. 96, pp. 5013-5019.
Kroes-Nijboer, A., Venema, P., Bouman, J., and van der Linden, E., 2009, “The critical aggregation concentration of β-lactoglobulin-based fibril formation,” Food Biophysics, Vol. 4, No. 2, pp. 59-63.
Kuipers, B. J. H., 2007, “Aggregation of peptides in soy protein isolate hydrolysates,” Ph.D dissertation, Wageningen University, The Netherlands.
Kuipers, B. J. H., Alting, A. C., and Gruppen, H., 2007, “Comparison of the aggregation behaviour of soy and bovine whey protein hydrolysates,” Biotechnology Advances, Vol. 25, pp. 606-610.
Kuwajima, K., 1989, “The molten globule state as a clue for understanding the folding and cooperativity of globular-protein structure,” Proteins: Structure, Function, and Genetics, Vol. 8, pp. 87-103.
Lopes, D. H. J., Meister, A., Gohlke, A., Hauser, A., Blume, A., and Winter, R., 2007,
“Memchanism of islet amyloid polypeptide fibrillation at lipid interfaces studied by infrared reflection absorption spectroscopy,” Biophysical Journal, Vol. 93, pp. 3132-3141.
Makin, O. S., Atkins, E., Sikorski, P., Johansson, J., and Serpell, L. C., 2005, “Molecular basis for amyloid fibril formation and stability,” Proceedings of the National Academy of Sciences, Vol. 102, No. 2, pp. 315-320.
Martinez, K. D., Sanchez, C. C., Rodriguez Patino, J. M., and Pilosof, A. M. R., 2009, “Interfacial and foaming properties of soy protein and their hydrolysates,” Food Hydrocolloids, Vol. 23, pp. 2149-2157.
Maruyama, N., Maruyama, Y., Tsuruki, T., Okuda, E., Yoshikawa, M., and Utsumi, S., 2003, “Creation of soybean beta-conglycinin beta with strong phatocytosis-stimulating activity,” Biochimica et Biophysica Acta, Vol. 1648, pp. 99-104.
Maruyama, Y., Maruyama, N., Mikami, B., and Utsumi, S. 2004, “Structure of the core region of the soybean beta-conglycinin alpha′ subunit,” Acta Crystallographica Section D, Vol. 60, pp. 289-297.
Mohsen Asghari, S., Khajeh, K., Moradian, F., Ranjbar, B., and Naderi-Manesh, H. 2004, “Acid-induced conformational changes in Bacillus amyloliquefaciens α-amylase: appearance of a molten globule like state,” Enzyme and Microbial Technology, Vol. 35, No. 1, pp. 51-57.
Moure, A., Domínguez and Parajó, J. C., 2006, “Antioxidant properties of ultrafiltration-recovered soy protein fractions from industrial effluents and their hydrolysates,” Process Biochemistry, Vol. 41, pp. 447-456.
MRSEC Interdisciplinary education group, University of Wisconsin Madison, “Octadecanethiol monolayer on silver,” <http://mrsec.wisc.edu/Edetc/nanolab/Agthiol>, accessed March 2011.
Naseem, F., Ahmad, B., Ashraf M. T., and Khan, R. H., 2004, “Molten globule-like folding intermediate of asialofetuin at acidic pH,” Biochimica et Biophysica Acta—Proteins & Proteomics, Vol. 1699, pp. 191-199.
Nielsen, L. E., Wall, R., and Adams, G., 1958, “Coalescence of liquid drops at oil-water interfaces,” Journal of Colloid Science, Vol. 13, No. 5, pp. 441-458.
Ogawa, S., Decker, E. A., and McClements D. J., 2003, “Influence of environmental conditions on the stability of oil in water emulsions containing droplets stabilized by lecithin-chitosan membranes,” Journal of Agricultural and Food Chemistry, Vol. 51, No. 18, pp. 5522-5527.
Park, E. Y., Morimae, M., Matsumura, Y., Nakamura, Y., and Sato, K., 2008, “Antioxidant activity of some protein hydrolysates and their fractions with different isoelectric points,” Journal of Agricultural and Food Chemistry, Vol. 56, pp. 9246-9251.
Peña-ramos, E. A. and Xiong, Y. L., 2002, “Antioxidant activity of soy protein hydrolysates in a liposomal system,” Journal of Food Science, Vol. 67, pp. 2952-2956.
Provencher, S. W. and Glockner, J., 1981, “Estimation of globular protein secondary structure from circular dichroism,” Biochemistry, Vol. 20, pp. 33-37.
Ruiz-Henestrosa, V. P., Sanchez, C. C., del Mar Yust, M., Pedroche, J., Milían, F., and Rodriguez Patino, J. M., 2007, “Limited enzymatic hydrolysis can improve the interfacial and foaming characteristics of β-conglycinin,” Journal of Agricultural and Food Chemistry, Vol. 55, pp. 1536-1545.
Ruiz-Henestrosa, V. P., Sánchez, C. C., Pedroche, J. J., Millán, F., and Rodriguez Patino, J. M., 2009, “Improving the functional properties of soy glycinin by enzymatic treatment. Adsorption and foaming characteristics,” Food Hydrocolloids, Vol. 23, pp. 377-386.
Ryan, M., McEvoy, E., McSweeney, S. L., O'Callaghan, D. M., and FitzGerald, R. J., 2008, “Thermal behaviour of emulsions manufactured with soy protein ingredients,” Food Research International, Vol. 41, pp. 813-818.
Sagis, L. M. C., de Ruiter, R, Rossier Miranda, F. J., de Ruiter, J., Schroen, K., van Aelst, A. C., Kieft, H., Boom, R., and van der Linden, E., 2008, “Polymer microcapsules with a fiber-reinforced nanocomposite shell,” Langmuir, Vol. 24, pp. 1608-1612.
Saio, K., Kamiya, M., and Watanabe, T., 1969, “Food processing characteristics of soybean-11S and soybean-7S proteins. 1. Effect of difference of protein components among soybean varieties on formation of tofu-gel,” Agricultural and Biological Chemistry, Vol. 33, No. 9, pp. 1301-1308.
Sambasivam, D., Liu, C. W., Jayaraman, M., Malar, E. J., and Rajadas, J., 2008, “Aggregation and conformational studies on a pentapeptide derivative,” Biochimica et Biophysica, Vol. 1784, No. 11, pp. 1659-1667.
Santiago, L. G., Maldonado-Valderrama, J., Martín-Molina, A., Haro-Pérez, C., García-Martínez, J., Martín-Rodríguez, A., Cabrerizo-Vilchez, M. A., and Gálvez-Ruiz, M. J., 2008, “Adsorption of soy protein isolate at air-water and oil-water interfaces,” Colloids and Surfaces A: Physicochemical and Engineering Aspects, Vol. 323, pp. 155-162.
Schladitz, C., Vieira, E. P., Hermel, H., and Mohwald, H., 1999, “Amyloid-β-sheet formation at the air-water interface,” Biophysical Journal, Vol. 77, pp. 3305-3310.
Sen, P., Ahmad, B., and Khan, R. H., 2008, “Formation of a molten globule like state in bovine serum albumin at alkaline pH,” European Biophysics Journal, Vol. 37, No. 8, pp. 1303-1308.
Sharma, D., Sharma, S., Pasha, S., and Brahmachari, S. K., 1999, “Peptide models for inherited neurodegenerative disorders: conformation and aggregation properties of long polyglutamine peptides with and without interruptions,” FEBS Letters, Vol. 456, pp. 181-185.
Shortwell, M. A. and Larkins, B. A., 1989, “The biochemistry and molecular biology of seed storage proteins,” In: The Biochemistry of Plants, Vol 15. Academic Press, New York, pp. 297-345.
Sugawara, T., Kuwajima, K., and Sugai, S., 1991, “Folding of staphylococcal nuclease A studied by equilibrium and kinetic circular dichroism spectra,” Biochemistry, Vol. 30, pp. 2698-2706.
Thomas, J. C., 1986, “The determination of log normal particle size distributions by dynamic light scattering,” Journal of Colloid and Interface Science, Vol. 117, pp. 187-192.
Van Stokkum, I. H. M., Spoelder, H. J. W., Bloemendal, M., Van Grondelle, R., and Groen, F. C. A., 1990, “Estimation of protein secondary structure and error analysis from CD spectra,” Analytical Biochemistry, Vol. 191, pp. 110-118.
Villar-Piqué, A., Sabaté, R., Lopera, O., Gibert, J, Tome, J. M., Santos, M., and Ventura, S., 2010, “Amyloid-Like Protein Inclusions in Tobacco Transgenic Plants,” PLoS ONE, Vol. 5, No. 10: e13625. doi:10.1371/joumal.pone.0013625
Whitmore, L. and Wallace, B. A., 2004, “Dichroweb, an online server for protein secondary structure analyses from circular dichroism spectroscopic data,” Nucleic Acids Research, Vol. 32, pp. W668-W673.
Wierenga, P. A., Egmond, M. R., Voragen, A. G. J., and de Jongh, H. H. J., 2006, “The adsorption and unfolding kinetics determines the folding state of proteins at the air-water interface and thereby the equation of state,” Journal of Colloid and Interface Science, Vol. 299, pp. 850-857.
Yee, J. J., Shipe, W. F., and Kinsella, J. E., 1980, “Antioxidant effects of soy protein hydrolysates on copper-catalyzed methyl linoleate oxidation,” Journal of Food Science, Vol. 45, pp. 1082-1083.

What is claimed is:

1. A composition, comprising:
an emulsion composing oil and water; and partially
hydrolyzed soy bean beta-conglycinin,
wherein the composition is one of a food composition, a food additive, a medical composition, at least one flavoring agent, at least one carbohydrate source, a composition for treating an essential fatty acid deficiency, a pharmaceutically acceptable carrier, or a combination thereof.

2. The composition of claim 1, wherein the degree of hydrolysis of the partially hydrolyzed soy bean beta-conglycinin is up to about 2.5%.

3. The composition of claim 1, wherein the partially hydrolyzed soy bean beta-conglycinin constitutes at least about 70% of the soy bean derived protein in the composition.

4. The composition of claim 1, wherein the soy bean beta-conglycinin is enzymatically hydrolyzed.

5. The composition of claim 1, wherein the soy bean beta-conglycinin is trypsinized.

6. The composition of claim 1, wherein the oil comprises omega-3 fatty acids.

7. The composition of claim 1, wherein the partially hydrolyzed soy bean beta-conglycinin constitutes less than about 5% w/v of the composition.

8. The composition of claim 1, wherein the soy bean beta-conglycinin is partially hydrolyzed to such an extent that the oil in water emulsion has an oxidative stability greater than and a physical stability at least equal to a corresponding oil in water emulsion prepared with a corresponding nonhydrolyzed soy bean beta-conglycinin.

9. The composition of claim 1, wherein the degree of hydrolysis of the partially hydrolyzed soy bean beta-conglycinin is up to about 1.5%.

10. The composition of claim 1, which is a food product composition containing at least one member selected from the group consisting of a flavorant, a colorant, a source of protein other than the partially hydrolyzed soy beta-conglycinin, and a source of carbohydrate.

11. The composition of claim wherein the partially hydrolyzed soy bean beta-conglycinin forms fibril sheets adsorbed to oil droplets in the emulsion.

12. The composition of claim 1, wherein the partially hydrolyzed soy bean beta-conglycinin increases the stability of the oil to oxidation as compared to a corresponding nonhydrolyzed soy bean beta-conglycinin.

13. The composition of claim 1, wherein the partially hydrolyzed soy bean beta-conglycinin forms a barrier at an oil/water interface that is less permeable to oxygen than a barrier formed by a corresponding nonhydrolyzed soy bean beta-conglycinin.

14. A method for preparing an emulsion composition, comprising: emulsifying a mixture including water, oil, and partially hydrolyzed soy bean beta-conglycinin, wherein the emulsion composition is one of a food composition, a food additive, a medical composition, at least one flavoring agent, at least one carbohydrate source, a composition for treating an essential fatty acid deficiency, a pharmaceutically acceptable carrier, or a combination thereof.

15. The method of claim 14, wherein the degree of hydrolysis of the partially hydrolyzed soy bean beta-conglycinin is up to about 2.5%.

16. The method of claim 15, wherein the partially hydrolyzed soy bean beta-conglycinin constitutes at least about 70% of the soy protein in the mixture.

17. The method of claim 14, wherein the soy bean beta-conglycinin is enzymatically hydrolyzed.

18. The method of claim 14, wherein the soy bean beta-conglycinin is trypsinized.

19. The method of claim 14, wherein the oil comprises omega-3 fatty acids.

20. The method of claim 14, wherein the partially hydrolyzed soy bean beta-conglycinin constitutes less than about 1% w/v of the mixture.
